(19) United States
(12) Patent Application Publication
(10) Pub. No.: US 2023/0158176 A1
(43) Pub. Date: May 25, 2023

(54) NEAR-INFRARED FLUORESCENT SMALL-MOLECULE PROBE, SYNTHESIS METHOD AND APPLICATION THEREOF

(71) Applicant: Linyi University, Linyi (CN)

(72) Inventors: Zongjin Qu, Linyi (CN); Han Li, Heze (CN); Zhen Wang, Zaozhuang (CN); Shusheng Zhang, Qingdao (CN)

(21) Appl. No.: 17/993,234

(22) Filed: Nov. 23, 2022

(30) Foreign Application Priority Data
Nov. 23, 2021 (CN) 2021113909120

(51) Int. Cl. A61K 49/00; A61B 5/00; A61N 5/06; A61K 41/00; A61P 35/00

(52) U.S. Cl. CPC A61K 49/0032; A61B 5/0071; A61N 5/0625; A61N 5/062; A61K 41/0052; A61P 35/00

(57) ABSTRACT

A near-infrared fluorescent small-molecule probe, a synthesis method thereof, and an application thereof are provided. The near-infrared fluorescent small-molecule probe is IR-780-F derived from IR-780, in which the basic structure of IR-780 is retained, an end of an alkyl carbon chain connected with a nitrogen (N) atom is changed by adding a trifluoromethyl group (—CF₃) at the end. The IR-780-F shows excellent performance in photostability, targeting cancer cells and fluorescence imaging, and has low toxicity. It can be used for targeting tumor tissues, in vivo near-infrared fluorescence imaging, and photothermal therapy, which realizes an integration of diagnosis and treatment.

### TECHNICAL FIELD

The disclosure relates to the field of biomedical technologies, and in particular to a near-infrared fluorescent small-molecule probe, a synthesis method thereof, and an application thereof.

### BACKGROUND

At present, surgical excision, radiotherapy and chemotherapy are important methods for clinical treatment of cancer, but the above methods have certain limitations, and the curative effect still needs to be improved. Therefore, it is urgent to develop new methods and new medicines for cancer treatment.

In recent years, emerging cancer treatment strategies include immunotherapy, gene therapy, photodynamic therapy, photothermal therapy (PTT) and so on. Specifically, the photothermal therapy has become a research hotspot in the field of tumor treatment because of its advantages of non-invasiveness, accurate space-time control, strong specificity and high efficiency of tumor destruction. Near-infrared (NIR) has good tissue penetration ability and high-resolution adjustability in time and space. During the photothermal therapy, the temperature change at the tumor location can be caused by the conversion of the optical energy of NIR into heat energy by photothermal medicines, and tumor cells can be effectively killed at a temperature above 42° C. Moreover, the heat resistance of the tumor cells is worse than that of normal cells; in this case, the increasing temperature can kill the tumor cells, thus avoiding obvious side effects on the normal cells.

When the photothermal agent in a ground state is irradiated by a light source and transitions to the S1 state, one part transitions back to the ground state in a form of radiation, and the other part returns to the ground state by transferring energy in the form of heat to the surrounding environment. All of laser transmission modes in the photothermal therapy are designed to uniformly increase the temperature in the tumor locations while preventing damage to surrounding healthy tissue. However, since the effective ablation of tumor requires destruction of each tumor cell, the photothermal therapy typically requires a center of the tumor to reach a higher temperature (≥50° C.) and a temperature gradient to make the edge of the tumor reach the treatment temperature. Therefore, it is required that the photothermal agents have a high photothermal conversion efficiency and excellent tumor targeting.

At present, researchers have developed many photothermal agents with the high photothermal conversion efficiency for tumor photothermal therapy, mainly including noble metal nanomaterials, carbon-based nanomaterials, metallic and non-metallic compound nanomaterials, and organic small-molecule dyes. Although nanoparticles have good photothermal conversion efficiency, the problems of difficult degradation in vivo, complex preparation process, potential long-term toxicity, and uneven size greatly limit the further clinical conversion of nano-photothermal materials, while the organic small-molecule dyes have natural advantages in the above aspects.

Most of the organic small-molecule photothermal agents reported in the existing literature need to use the “nanoization” strategy to assist in playing their photothermal therapeutic performance, however, it makes the medical system face common limitations of the “nanomaterial” system, and it is difficult to further carry out the clinical translation. IR-780 has a natural tumor-targeting function, but its photothermal performance needs to be improved, and the administration mode in the tumor-bearing mouse model is intratumoral injection.

The disclosure significantly improves the photothermal conversion effects of the IR-780 by a modification of a trifluoromethyl group (—CF₃), and achieves tumor targeting photothermal treatment in tumor-bearing mice via tail vein injection.

### SUMMARY

In response to the above problems in the related art, the disclosure provides a near-infrared fluorescent small-molecule probe, a synthesis method thereof, and an application thereof, in particular to a near-infrared fluorescent small-molecule probe for targeting breast carcinoma therapy, a synthesis method thereof, and an application thereof.

The disclosure provides the synthesis method of the near-infrared fluorescent small-molecule probe, including the following steps:

step 1, adding carbon tetrabromide (CBr₄, also referred to as tetrabromomethane) and triphenylphosphine (PPh₃) into a 100 milliliters (mL) three-necked flask to obtain a solution, placing the 100 mL three-necked flask added with the solution in an ice-water mixture to make the solution cool down to 0 Celsius degree (° C.), and then slowly adding a 3,3,3-trifluoro-1-propanol (C₃H₅F₃O) reagent to the 100 mL three-necked flask added with the solution;

step 2, obtaining a first reaction solution until the solution in the step 1 to be a yellow viscous liquid after adding the 3,3,3-trifluoro-1-propanol reagent; heating and refluxing the 100 mL three-necked flask at 60° C. for 1 hour (h) after moving the 100 mL three-necked flask with the first reaction solution to a room temperature; cooling down the 100 mL three-necked flask and setting up a distillation apparatus, heating the distillation apparatus to vaporize a vaporized product, heating up to 100° C. to vaporize a liquid, and continuing to heat until no evaporated liquid to obtain a first colorless liquid compound;

step 3, dissolving the first colorless liquid compound, 2,3,3-trimethylindolenine (C₁₁H₁₃N) and potassium iodide (KI) into acetonitrile to heat to 150° C. in a closed container (also referred to as a hydrothermal synthesis reactor) to obtain a first mixture, stirring the first mixture overnight for reaction to obtain a second reaction solution;

step 4, determining a reaction extent of the second reaction solution by using thin layer chromatography; the thin layer chromatography displaying less surplus of raw materials and the second reaction solution changing from light yellow to salmon pink; filtering the second reaction solution to obtain a first filtrate and a filter cake, washing the filter cake with the acetonitrile to obtain a second filtrate; concentrating the first filtrate and the second filtrate to obtain a first concentrated product, and performing silica gel column chromatography on the first concentrated product to obtain a second compound;

step 5, dissolving the second compound and 2-chloro-1-formyl-3-hydroxymethylenecyclohexene (C₈H₉O₂Cl) into a mixed solution of butyl alcohol and methylbenzene to obtain a second mixture; stirring the second mixture at 110° C. overnight for reaction, to obtain a third reaction solution;

step 6, determining a reaction extent of the third reaction solution by using thin layer chromatography; the thin layer chromatography displaying no surplus of the raw materials and generating a new sample spot on a thin layer chromatography plate; stopping reaction of the third reaction solution, concentrating and drying the third reaction solution to obtain a second concentrated product, and purifying the second concentrated product by the silica gel column chromatography to obtain a targeting product IR-780-F.

In an illustrated embodiment of the disclosure, in the step 1, an addition amount of the carbon tetrabromide is 13.95 grains (g) with a molar mass of 42.08 millimoles (mmol); an addition amount of the triphenylphosphine is 11.04 g with a molar mass of 42.08 mmol; and an addition amount of the 3,3,3-trifluoro-1-propanol is 4.0 g with a molar mass of 35.07 mmol.

In an illustrated embodiment of the disclosure, in the step 2, a mass of the first colorless liquid compound is 0.75 g, taking a percentage of 12.1% in the first reaction solution.

In an illustrated embodiment of the disclosure, in the step 3, an addition amount of the first colorless liquid compound is 0.65 g with a molar mass of 4.24 mmol; an addition amount of the 2,3,3-trimethylindolenine is 0.45 g with a molar mass of 2.83 mmol; an addition amount of the potassium iodide is 0.47 g with a molar mass of 2.83 mmol; a volume of the acetonitrile is 20 mL.

In an illustrated embodiment of the disclosure, in the step 4, an developing reagent of the thin layer chromatography is a volume ratio of petroleum ether:ethyl acetate being 10:1; a condition for the silica gel column chromatography is a volume ratio of the petroleum ether: the ethyl acetate being a range from 100:1 to 5:1; and a mass of the second compound is 60 milligrams (mg), taking a percentage of 8.48% in the second reaction solution.

In an illustrated embodiment of the disclosure, in the step 5, an addition amount of the second compound is 0.02 g with a molar ratio of 0.08 mmol; an addition amount of the 2-chloro-1-formyl-3-hydroxymethylenecyclohexene is 0.01 g with a molar ratio of 0.04 mmol; a volume ratio of the butyl alcohol: the methylbenzene in the mixed solution is 7:3, and a total volume of the mixed solution of the butyl alcohol and the methylbenzene is 2 mL.

In an illustrated embodiment of the disclosure, in the step 6, an developing reagent of the thin layer chromatography is a volume ratio of the petroleum ether: the ethyl acetate being 10:1; and a condition for the purifying by the silica gel column chromatography is a volume ratio of the petroleum ether: the ethyl acetate being 1:1.

Another object of the disclosure is to provide an application of the near-infrared fluorescent small-molecule probe synthesized by the above method.

In an illustrated embodiment of the disclosure, the near-infrared fluorescent small-molecule probe is IR-780-F and a molecular formula of the IR-780-F is C₃₆H₃₈F₆ClN₂I with a structural formula shown as follows:

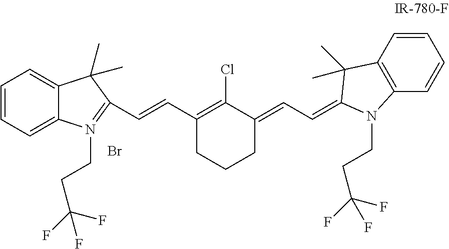

Another object of the disclosure is to provide the application of the near-infrared fluorescent small-molecule probe to prepare a medicine for targeting tumor tissues, in vivo near-infrared fluorescence imaging, and tumor photothermal therapy.

Another object of the disclosure is to provide a testing kit for targeting tumor tissues, in vivo near-infrared fluorescence imaging, tumor photothermal and targeting breast carcinoma, and the testing kit includes the near-infrared fluorescent small-molecule probe.

Combining the above technical solutions, the advantages and beneficial effects of the disclosure are as follows.

The near-infrared fluorescent small-molecule probe provided by the disclosure is derived from an IR-780, of which a molecular formula is C₃₆H₄₄ClN₂I, retaining the basic structure of IR-780, changing an end of alkyl carbon chain connected with a nitrogen (N) atom, replacing a methyl group (—CH₃) with a trifluoromethyl group (—CF₃), which can be used for targeting tumor tissue, in vivo near-infrared fluorescence imaging, and tumor photothermal therapy. Furthermore, the near-infrared fluorescent small-molecule probe of the disclosure has a lower toxicity and enhances capability of the photothermal conversion.

According to the disclosure, the IR-780 is innovatively modified with the trifluoromethyl group, and it is found that the modified IR-780-F performs excellent photothermal agent performance than the IR-780. Especially, the administration mode of intratumoral injection into tumor-bearing mice performs well in tumor photothermal treatment. The high tumor targeting and high photothermal properties of the IR-780-F are the outstanding advantages of the disclosure. Compared with those photothermal medicines without the capacity of tumor targeting, the outstanding advantages are especially conductive to the photothermal therapy research for scattered and undetected tumors.

The disclosure obtains the IR-780 derivative with good stability, strong targeting capacity, and significantly higher photothermal conversion capacity through the —CF₃ group modification, which provides preclinical experimental research for the application of the IR-780 derivative in photothermal therapy of tumors, thus promoting further clinical studies of organic small-molecule photothermal agents.

### BRIEF DESCRIPTION OF DRAWINGS

In order to explain technical solutions of embodiments of the disclosure more clearly, the followings will briefly introduce attached drawings required in the embodiments. Apparently, the attached drawings in the following descriptions are only some of the embodiments of the disclosure. For those skilled in the art, other drawings can be obtained according to the attached drawings without creative effort.

FIG. 1 is a flowchart of a synthesis method of a near-infrared fluorescent small-molecule probe according to an embodiment of the disclosure.

FIG. 2 is a schematic diagram of the synthesis method of the near-infrared fluorescent small-molecule probe according to the embodiment of the disclosure.

FIGS. 3A-3B show spectral properties of IR-780-F in different solvents according to an embodiment of the disclosure.

FIGS. 4A-4B show photothermal effects in vitro of IR-780 and IR-780-F in different concentrations according to an embodiment of the disclosure.

FIG. 5 is a schematic diagram of cytotoxicity of the IR-780 and the IR-780-F in the different concentrations according to the embodiment of the disclosure.

FIG. 6 is a schematic diagram of a distribution of the IR-780-F in mice according to the embodiment of the disclosure.

FIGS. 7A-7B show in vivo photothermal effects of the IR-780-F according to the embodiment of the disclosure.

FIGS. 8A-8E show in vivo therapeutic effects of the IR-780-F according to the embodiment of the disclosure.

### DETAILED DESCRIPTION OF EMBODIMENTS

In order to make the objects, the technical solutions and the advantages of the disclosure more clear, the disclosure is further described in detail below in combination with the embodiments. It should be understood that the illustrated embodiments described herein are only used to explain the disclosure, and are not intended to limit the disclosure.

In view of the problems existing in the related art, the disclosure provides a near-infrared fluorescent small-molecule probe, a synthesis method thereof, and an application thereof. The disclosure is described in detail below in combination with the attached drawings.

In an embodiment of the disclosure, the near-infrared fluorescent small-molecule probe is IR-780-F.

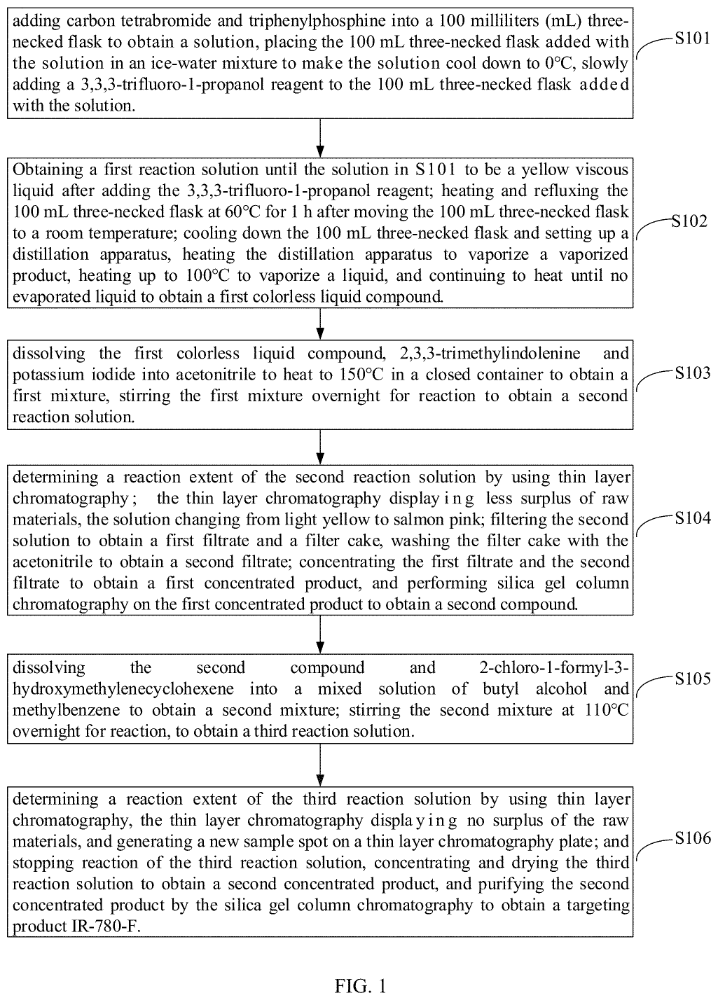

As shown in FIG. 1, an embodiment of the disclosure provides a synthesis method of the near-infrared fluorescent small-molecule probe, including following steps:

S5101, adding carbon tetrabromide (CBr₄, also referred to as tetrabromomethane) and triphenylphosphine (PPh₃) into a 100 milliliters (mL) three-necked flask to obtain a solution, placing the 100 mL three-necked flask added with the solution in an ice-water mixture to make the solution cool down to 0 Celsius degree (° C.), and then slowly adding a 3,3,3-trifluoro-1-propanol (C₃H₅F₃O) reagent to the 100 mL three-necked flask added with the solution;

S102, obtaining a first reaction solution until the solution in the S101 to be a yellow viscous liquid after adding the 3,3,3-trifluoro-1-propanol reagent; heating and refluxing the 100 mL three-necked flask at 60° C. for 1 hour (h) after moving the 100 mL three-necked flask with the first reaction solution to a room temperature; cooling down the 100 mL three-necked flask and setting up a distillation apparatus, heating the distillation apparatus to vaporize a vaporized product, heating up to 100° C. to vaporize a liquid, and continuing to heat until no evaporated liquid to obtain a first colorless liquid compound 1;

S103, dissolving the first colorless liquid compound 1, 2,3,3-trimethylindolenine (C₁₁H₁₃N) and potassium iodide (KI) into acetonitrile to heat to 150° C. in a closed container to obtain a first mixture, stirring the first mixture overnight for reaction to obtain a second reaction solution;

S104, determining a reaction extent of the second reaction solution by using thin layer chromatography; the thin layer chromatography displaying less surplus of raw materials and the second reaction solution changing from light yellow to salmon pink; filtering the second reaction solution to obtain a first filtrate and a filter cake, washing the filter cake with the acetonitrile to obtain a second filtrate; concentrating the first filtrate and the second filtrate to obtain a first concentrated product, and performing silica gel column chromatography on the first concentrated product to obtain a second compound 2;

S105, dissolving the second compound 2 and 2-chloro-1-formyl-3-hydroxymethylenecyclohexene (C₈H₉O₂Cl) into a mixed solution of butyl alcohol and methylbenzene to obtain a second mixture; stirring the second mixture at 110° C. overnight for reaction to obtain a third reaction solution;

S106, determining a reaction extent of the third reaction solution by using thin layer chromatography; the thin layer chromatography displaying no surplus of the raw materials and generating a new sample spot on a thin layer chromatography plate; stopping reaction of the third reaction solution, concentrating and drying the third reaction solution to obtain a second concentrated product, and purifying the second concentrated product by the silica gel column chromatography to obtain a targeting product IR-780-F.

The technical solutions of the disclosure are further described in combination with the illustrated embodiments.

### Embodiment

1. Overview of the Disclosure

The disclosure relates to a small-molecule probe for targeting breast carcinoma therapy and dual-modality imaging and photothermal therapy thereof. The small-molecule probe for dual-modality imaging and photothermal therapy provided by the disclosure is derived from IR-780, retaining the basic structure of the IR-780, changing an end of alkyl carbon chain connected with a nitrogen (N) atom, and adding a trifluoromethyl group (—CF₃) at the end. Therefore, the small-molecule probe can be used for targeting tumor tissues, in vivo near-infrared fluorescence imaging, and tumor photothermal therapy, and enhances photothermal conversion effects.

2. Design of the Disclosure

Experience has shown that in medicine, when a compound is introduced with fluorine atoms or fluorine-containing groups (especially a trifluoromethyl group, also referred to as the —CF3 group), of which electronic and analog effects change a distribution of electron density inside the molecule, affects acid-base property of internal structure of the compound, which in turn changes an activity of the compounds, and increases liposolubility of the compound. The fluorine atom replaces the hydrogen atom in the compound, the solubility of the ester-like compound in biological membranes is enhanced, which promotes transmission speed of its absorption in the organism and changes physiological effect. Therefore, many fluorine-containing compounds have the advantages of less dosage, lower toxicity, higher efficacy and stronger metabolic capability than non-fluorine-containing compounds in pharmaceutical properties of medicine and pesticide.

The structural modification of the IR-780 tends to make the molecular targeting reduced. In order to retain the molecular targeting, the disclosure only replaces the original methyl function group in the molecule with the trifluoromethyl group with relatively inert chemical activity, and a bond length of carbon-fluorine (C—F) bond increases little compared with that of carbon-hydrogen (C—H) bond, and the geometric configurations of the two molecules are similar. In the molecular structure, the basic structure of IR-780 is retained, and the end of the alkyl carbon chain end connected with the nitrogen atom is changed and the trifluoromethyl group is added to obtain the IR-780 derivative, i.e., IR-780-F, which is used for the preclinical experimental research of an application of the IR-780 in tumor photothermal therapy with a view to promoting further clinical researches of organic small-molecule photothermal agents.

2.1 Synthesis Steps

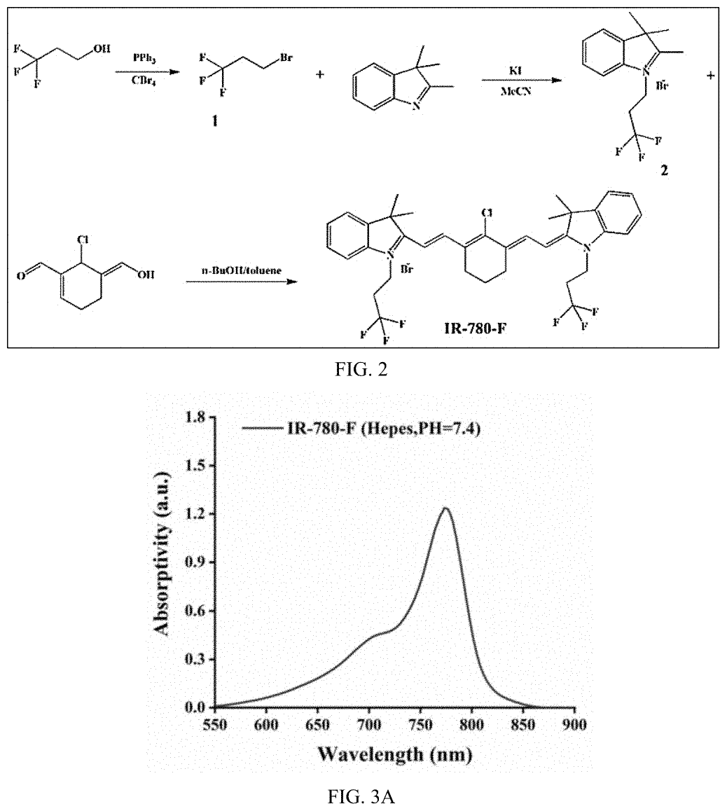

The synthesis steps of the IR-780-F molecule probe are shown in FIG. 2, which is specified as follows.

13.95 grains (g) carbon tetrabromide with a molar mass of 42.08 millimoles (mmol) and 11.04 g triphenyl phosphorus with a molar mass of 42.08 mmol are added into a 100 milliliters (mL) three-necked flask to obtain a solution. The 100 mL three-necked flask added with the solution is placed in an ice-water mixture to make the solution cool down to 0 Celsius degree (° C.), and then 4.0 g 3,3,3-trifluoro-1-propanol reagent with a molar mass of 35.07 mmol is slowly added into the 100 mL three-necked flask added with the solution until the solution mixed with the 3,3,3-trifluoro-1-propanol reagent to be a yellow viscous liquid to thereby obtain a first reaction solution. The 100 mL three-necked flask is heated and refluxed at 60° C. for 1 hour (h) after moving the 100 mL three-necked flask with the first reaction solution to a room temperature. The 100 mL three-necked flask is cooled down and a distillation apparatus is set up, the distillation apparatus is heated to vaporize a vaporized product, then heated up to 100° C. to vaporize a liquid, and continuously heated until no evaporated liquid to obtain 0.75 g first colorless liquid compound 1, which takes a percentage of 12.1% in the first reaction solution.

0.65 g first colorless liquid compound 1 with a molar mass of 4.24 mmol, 0.45 g 2,3,3-trimethylindolenine with a molar mass of 2.83 mmol and 0.47 g potassium iodide with a molar mass of 2.83 mmol are dissolved into 20 mL acetonitrile to heat to 150° C. in a closed container (also referred to a hydrothermal synthesis reactor) to obtain a first mixture. The first mixture is stirred overnight for reaction to obtain a second reaction solution. A reaction extent of the second reaction solution is determined by using thin layer chromatography with a developing reagent of a volume ratio of petroleum ether:ethyl acetate being 10:1, the thin layer chromatography displaying less surplus of raw materials and the second reaction solution changing from light yellow to salmon pink. The second reaction solution is filtered to obtain a first filtrate and a filter cake, the filter cake is rinsed with the acetonitrile to obtain a second filtrate, and the first filtrate and the second filtrate is concentrated to obtain a first concentrated product, and silica gel column chromatography with a condition of a volume ratio of the petroleum ether: the ethyl acetate being a range from 100:1 to 5:1 is performed on the first concentrated product to obtain 60 milligrams (mg) second compound 2, which takes a percentage of 8.48% in the second reaction solution.

0.02 g second compound 2 with a molar mass of 0.08 mmol and 0.01 g 2-chloro-1-formyl-3-hydroxymethylenecyclohexene with a molar mass of 0.04 mmol are dissolved into 2 mL mixed solution of butyl alcohol and methylbenzene with a volume ratio of the butyl alcohol: the methylbenzene being 7:3 to obtain a second mixture. The second mixture is stirred at 110° C. overnight for reaction to obtain a third reaction solution. A reaction extent of the third reaction solution is determined by using the thin layer chromatography with the developing reagent with a volume ratio of the petroleum ether: the ethyl acetate being 10:1, the thin layer chromatography displaying no surplus of the raw materials and generating a new sample spot on a thin layer chromatography plate. The reaction of the third reaction solution is stopped, the third reaction solution is concentrated and dried to obtain a second concentrated product, and the second concentrated product is purified by the silica gel column chromatography with a condition of a volume ratio of the petroleum ether: the ethyl acetate is 1:1 to obtain a targeting product IR-780-F.

2.2 Molecular Spectrum Test

A certain mass of the IR-780-F sample is weighed and dissolved in dimethyl sulfoxide (DMSO) to obtain a solving solution, and the quantitative solving solution is added to 2-[4-(2-hydroxyethyl)-1-piperazinyl]-ethanesulfonic acid (HEPES, also referred to as C₈H₁₈N₂O₄S) buffer solution to form a solution with a concentration of 10.0 micromoles per liter (LM) (containing 10% dimethyl sulfoxide) and an absorption spectrum of the IR-780-F sample is measured. Ultraviolet absorption spectrum is obtained by Agilent Ultraviolet-Vis spectrophotometer with a scanning wavelength range from 500 nanometers (nm) to 900 nm at a sweep rate of 1002 nanometers per minute (nm/min). Furthermore, addition quantitative solving solution is respectively added to methanol, acetonitrile, phosphate buffered saline (PBS buffer solution), HEPES buffer solution, bovine serum and adult serum to form a solution with a concentration of 10.0 μM (containing 10% dimethyl sulfoxide) to test an emission spectrum of the IR-780-F sample. Hitachi F-4600 Fourier transform infrared (FT-IR) spectrometer is used to obtain FT-IR emission spectrum, of the scanning wavelength is from 750 nm to 900nm, an excitation wavelength λex is 740 nm, an excitation slit width is 5 nm, and an emission slit width is 5 nm.

2.3 In Vitro Photothermal Efficiency of the IR-780-F

To evaluate in vitro thermogenesis of IR-780 solution and IR-780-F solution, 10 millimoles per litter (mM) IR-780-F solution dissolved in the dimethyl sulfoxide is used, and the IR-780-F solution is respectively diluted to the concentrations of 0.2 milligrams per milliliters (mg/mL), 0.4 mg/mL, 0.8 mg/mL, and 1.2 mg/mL with the phosphate buffered saline and these IR-780-F solutions and the phosphate buffered saline are evaluated. The IR-780 solution is prepared as above.

Temperature changes of the IR-780-F solutions and the phosphate buffered saline and temperature changes of the IR-780 solutions and the phosphate buffered saline are respectively recorded by using an infrared thermal imaging camera (a model of FLIR-E6). The phosphate buffered saline as a control group, 0.2 mg/mL of the IR-780-F solution and 0.2 mg/mL of the IR-780 solution, 0.4 mg/mL of the IR-780-F solution and 0.4 mg/mL of the IR-780 solution, 0.8 mg/mL of the IR-780-F solution and 0.8 mg/mL of the IR-780 solution, 1.2 mg/mL of the IR-780-F solution and 1.2 mg/mL of the IR-780 solution are all exposed to 808 nm laser radiation with a power density of 2.0 Watt per square centimeter (W/cm²) to be measured and recorded the temperature changes.

3. Cell Experiments

3.1 In Vitro Evaluation of Cytotoxicity

Respective toxicity of the IR-780-F solution and the IR-780 solution with concentrations from 10 μM to 60 μM on murine mammary carcinoma cells (also referred to as 4T1 cells) is measured by cell counting kit-8 (CCK-8). Incubated 4T1 cells are collected and centrifuged to prepare cell suspensions, and a cell concentration is adjusted to 8×10⁴ cells per milliliter. The 4T1 cells are transferred to 96-well plates, and 100 microliters (μL) 4T1 cells are added to each well, so that a density of the cells to be tested is about 8000 cells per well, and the number of the 96-well plates for experiment is two. The two 96-well plates inoculated with the cells to be tested are placed in an incubator at 37° C. for 12 h. One plate is incubated with the IR-780-F solutions with a concentration gradient from 10 μM to 60 μM for 1 h; the other plate is incubated with the IR-780 solutions with a concentration gradient from 10 μM to 60 μM for 1 h. After the end of incubation, CCK-8 reagent (a volume of 10 μL, a concentration of 5 mg/mL, i.e. 0.5% CCK-8 reagent) is immediately added to the each well and the two 96-well plates continue to incubate for 40 min. Finally, absorptivity and intensity of the each well are tested by an enzyme calibration, and cell viability of the control group is taken as 100%, and the cell viability of the test groups are taken as the percentage of the control group.

4. Animal Experiments

4.1 Tumor Model

The incubated 4T1 cells are collected by centrifugation, rinsed three times with PBS buffer solution to prepare cell suspensions, which are used a cell counter to count cell number. The cell suspensions are respectively diluted to desired concentrations with the PBS buffer solution. The pretreated 4T1 cells (about 1×10⁶ per 100 μL) are injected subcutaneously into right hind legs of BALB/c Nude mice and tumor model mice are obtained after about one week.

4.2 In Vivo Imaging of the IR-780-F in the Tumor-Bearing Mice

In order to determine an optimal time for in vivo photothermal treatment with the IR-780-F, tissue permeability and distribution of the IR-780-F in tumor-bearing mice are examined by means of a whole animal near infrared (NIR) imaging system. An injection of the IR-780-F (containing 12.4% dimethyl sulfoxide) is prepared with PBS buffer solution. When the tumors in the tumor-bearing mice grow to a certain volume, 4 milligrams per kilogram of the tumor-bearing mice (mg/kg) IR-780-F is injected into the tumor-bearing mice via tail vein. The anesthetized nude mice are placed into a in vivo imaging apparatus (also referred to as living imaging apparatus) and subjected to fluorescence imaging at 0 h, 2 h, 4 h, 8 h, 12 h, 24 h, 48 h, 72 h, 96 h and 120 h. Five days later, the nude mice are dissected and major organs including intestine, lung, spleen, kidney, heart, stomach and tumor parts are taken out. The organs are placed in the living imaging apparatus to observe fluorescence distribution of each organ.

4.3 Photothermal Efficiency of the IR-780-F at Tumor Location

The injection of the IR-780-F (containing 12.4% dimethyl sulfoxide) is prepared with PBS buffer solution for intratumoral injection. After 12 hours, the nude mice are anesthetized, imaged using a photothermal imager, and temperature changes are recorded. The tumor locations of the mice are irradiated with a laser for 4 minutes, and then the photothermal imaging is again performed on the corresponding tumor locations to record the corresponding temperature.

4.4 Photothermal Treatment Effect

After successful tumor loading, mice with an average tumor diameter of about 10×10 mm are randomly divided into four groups (5 mice in each group): (1) control group: intratumoral injection of 0.1 mL of saline; (2) laser group: intratumoral injection of 0.1 mL of saline followed by a laser radiation (a wavelength of 808 nm, 2.0 W/cm², 4 min); (3) IR-780-F group: intratumoral injection of 0.1 mL (corresponding to 80 micrograms abbreviated μg) of the IR-780-F solution without the laser radiation; (4) IR-780-F+laser group: intratumoral injection of 80 μg of the IR-780-F solution followed by the laser radiation (a wavelength of 808 mn, 2.0 W/cm², 4 min). The mice are anesthetized after administration of the medicine, and the tumor locations of the mice are placed under the laser radiation. When the laser irradiated the tumor locations of the mice, the temperature of the tumor locations under the laser radiation are simultaneously recorded when measured by the thermal infrared imager.

5. Results and Discussions

5.1 Spectral Properties of the IR-780-F in Different Solvents

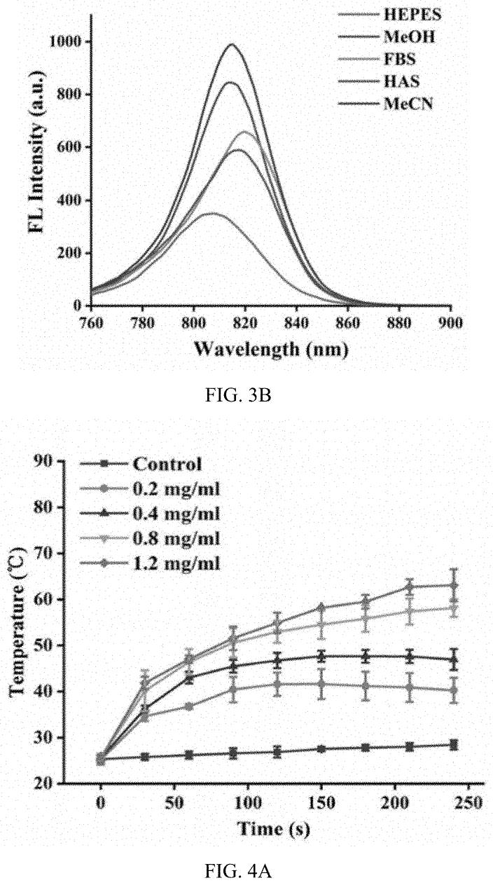

The photophysical properties of the IR-780-F are investigated. As shown in FIG. 3A, the maximum absorption wavelength of the IR-780-F in HEPES is 773 nm. And as shown in FIG. 3B, the maximum emission wavelengths of the IR-780-F in HEPES, MeOH, FBS, HAS and MeCN are respectively 804 nm, 814 nm, 820 nm, 818 nm and 816 nm, which are all falling within near-infrared spectral range. It has been calculated that the Stoke shift value, corresponding to the difference between the maximum emission wavelength and the maximum absorption wavelength is 38 nm, which is more conductive to the separation of the fluorescence generated by the IR-780-F in organisms from the background fluorescence and excitation wavelength, to avoid background interference signals in developing and to improve sensitivity of the fluorescence imaging. Red shift of the IR-780-F emission wavelength is observed in the bovine serum or the adult serum compared with that in other solvents, which due to the fact that some proteins in the serum combined with the IR-780-F to change the spectral properties of the molecule.

5.2 In Vitro Photothermal Efficiency of the IR-780-F

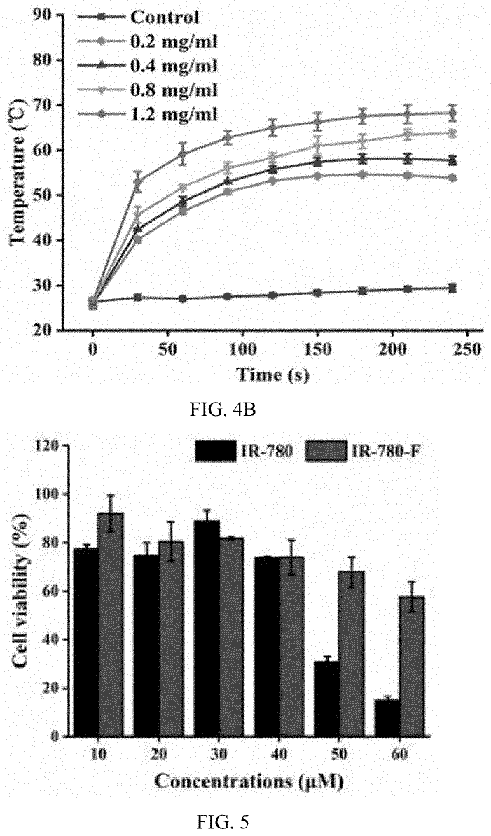

In order to investigate the potential of the IR-780-F as a photothermal agent, photothermal performance of the IR-780-F is systematically investigated by using the photothermal conversion effect of the IR-780 as a control. The respective temperature changes of the IR-780-F solution and the IR-780 solution under the 808 nm laser radiation are recorded. FIG. 4A shows the photothermal curves of the IR-780 under the 808 nm laser radiation. The temperature of the IR-780 solution increases rapidly with an increase of the concentration of the solution, with the highest temperature of 69.3° C. As shown in FIG. 4B, the temperature of the IR-780-F solution increases rapidly with an increase of the concentration of the solution under the 808 nm laser radiation with the highest temperature of 69.3° C. The photothermal curves of different concentrations of the IR-780-F solutions (the control group, 0.2 mg/mL, 0.4 mg/mL, 0.8 mg/mL, and 1.2 mg/mL) under the 808 nm laser radiation at a power of 2.0 W/cm² are also shown in FIG. 4B. However, the temperature of the PBS does not exhibit a significant increase. Therefore, it is concluded that the IR-780-F designed by the disclosure is an effective photothermal conversion agent for photothermal treatment and has better photothermal conversion effect than that of the IR-780.

5.3 Cytotoxicity of the IR-780-F

A magnitude of the toxicity to cancer cells is also one of the important properties of the of the organic small-molecule developers targeting tumor cells. The toxicity of the IR-780-F solutions in different concentrations on the 4T1 cells is detected by the CCK-8, with that of the IR-780 solutions as the control group. As shown in FIG. 5, the cell viability of the 4T1 cells decreases with the increase of the concentrations of the small-molecule probe when incubated with the IR-780 solutions or the IR-780-F solutions within the concentration range from 10 μM to 60 μM for 1 h.

5.4 Distribution of the IR-780-F in Mice

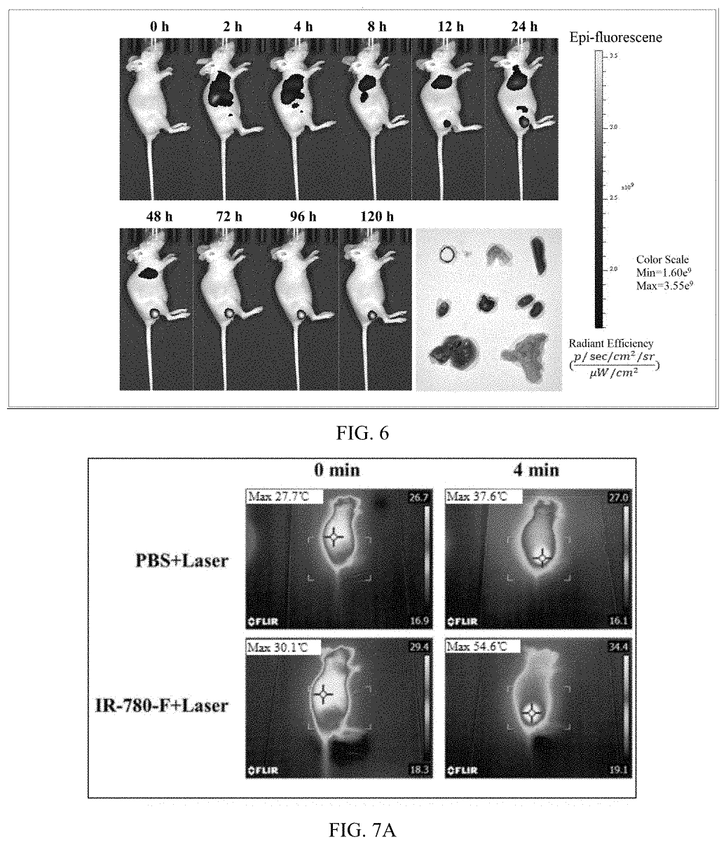

In order to determine the optimal timing of the IR-780-F for photothermal treatment in the mice, the disclosure detects the tissue permeability and medicine distribution of the IR-780-F in the tumor-bearing mice by the near-infrared in vivo imaging technology. The IR-780-F injection is injected into the 4T1 tumor-bearing nude mice via the tail vein, and the mice are anesthetized at different time intervals and placed in the living animal imaging system to observe the distribution of the near-infrared fluorescence in vivo. As shown in FIG. 6, the fluorescence signals in the tumor locations gradually increase with the increase of time, and the IR-780-F solution injected in the first 8 h is mainly concentrated in the heart, lung and thorax of the mice. With the increase of time, IR-780-F in the body repeats cycles, and gradually accumulates in the tumor locations, fluorescence intensity of tumor locations gradually increases, significantly higher than other organs and tissues. After 5 days, when the background fluorescence is removed, it is found that the fluorescence in the mice is mainly concentrated in the tumor location, and there is almost no fluorescence in other locations. The experimental results indicate that the IR-780-F has significant tumor targeting accumulation.

According to the experimental requirements and animal handling guidelines, the mice are euthanized and major organs and tumors of the mice are dissected and isolated. Fluorescence is mainly concentrated at the tumor location and there is weak fluorescence in the lungs, due to a small amount of the IR-780-F concentrating in the lungs and not completely metabolized. The experimental results after the dissection are consistent with those obtained from in vivo fluorescence imaging, further demonstrating that the IR-780-F has the property of tumor locations targeting, and the fluorescence is accumulated at the tumor locations and not easily metabolized out of body, which is suitable for the near-infrared fluorescence imaging of the tumor locations.

5.5 In Vivo Photothermal Effect of the IR-780-F

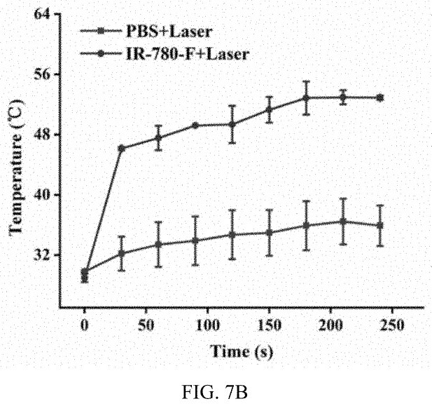

As shown in FIG. 7, thermal images and temperature changes in the tumor location are recorded with an infrared thermal imaging camera. After near-infrared laser radiation, surface temperature the tumor locations increases significantly to 52.9±0.2° C. in the IR-780-F+laser radiation group, but slightly increases to 35.9±2.7° C. in the PBS+laser radiation group. IR-780-F induces high photothermal effects in vivo after exposure to the near-infrared laser radiation, thus indicating that the IR-780-F molecule is promising in the photothermal treatment (PPT) in vivo.

5.6 In Vivo Therapeutic Effect of the IR-780-F

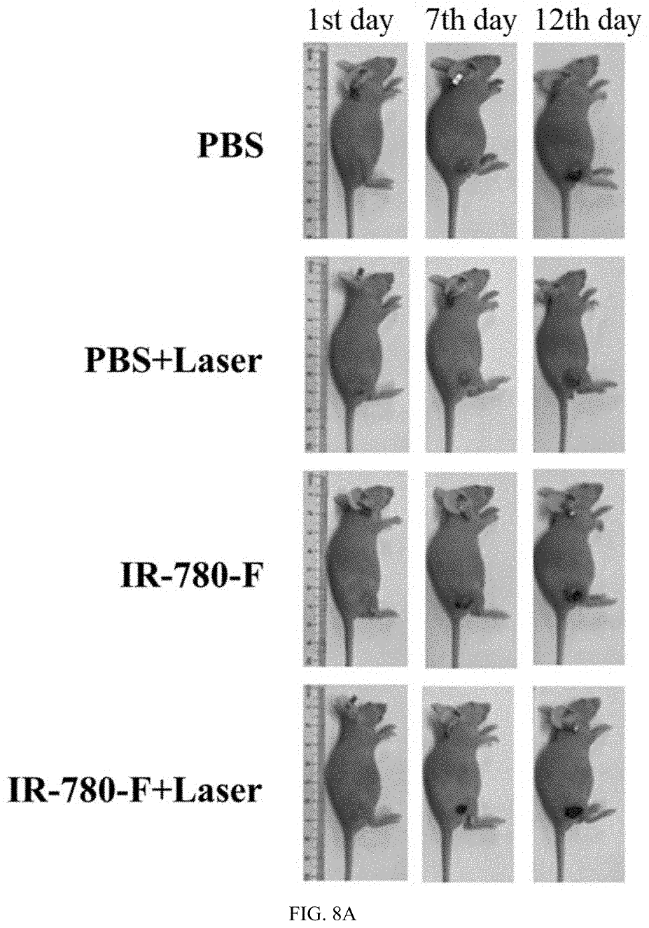

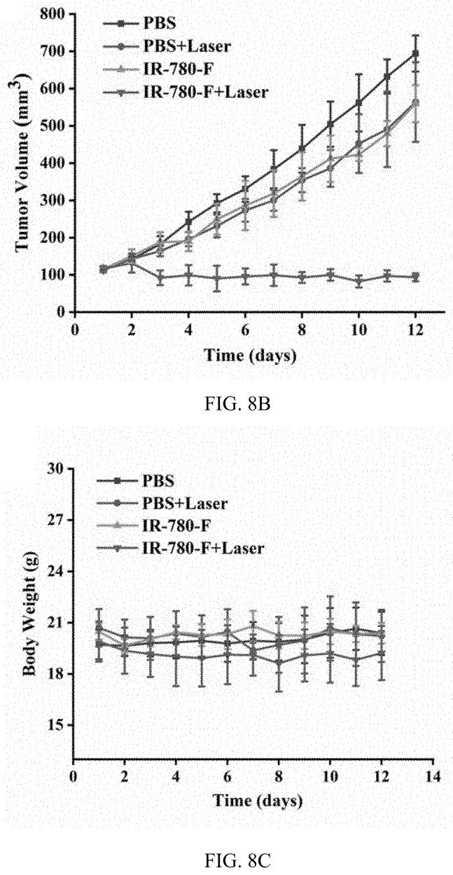

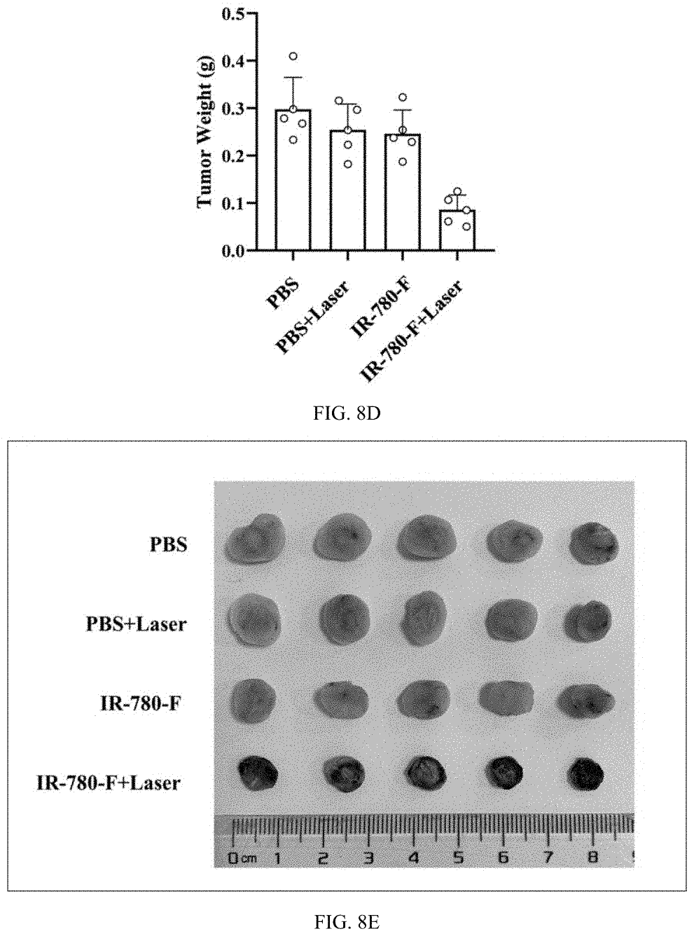

The disclosure tests the tumor treatment effect of the IR-780-F as a photothermal agent. FIG. 8A shows the tumors in four groups of the tumor-bearing mice at the first day, the 7^{th} day and the 12^{th} day after the treatment. FIG. 8B shows tumor growth curves of the four groups. The tumor growth is significantly inhibited by the IR-780-F+laser radiation (probability (P)<0.01), and the mean tumor size is 94±11 cubic millimeters (mm³) at the 12^{th} day of the photothermal treatment. In contrast, the IR-780-F group does not show a significant inhibition of tumor growth compared with the laser radiation alone and the control PBS group (P>0.05), and the tumor sizes of the three groups are 559±49 mm³, 564±107 mm³ , and 694.046±48.45 mm³ on the 12^{th} day, respectively (P>0.05) (as shown in FIG. 8C). As shown in FIG. 8D, the tumor-bearing mice are sacrificed at the 12^{th} day to obtain the tumors, and the tumors are weighed by an electronic balance, and the tumor volumes in the IR-780-F+laser radiation group are significantly smaller than those in the other three groups, which is also illustrated by an anatomical diagram of the tumor in FIG. 8E.

In the descriptions of the disclosure, unless otherwise stated, “multiple” means two or more. The terms indicating an orientation or a position relationship, such as “up”, “down”, “left”, “right”, “inside”, “outside”, “front end”, “back end”, “head”, “tail”, etc. are based on the attached drawings, and are only convenient for describing the disclosure and simplifying the descriptions, rather than indicating or implying that the device or component referred to must have a specific orientation, or be constructed and operated in a specific orientation. Therefore, the above terms cannot be understood as a limitation for the disclosure. In addition, the terms “first”, “second”, “third”, etc. are only used to describe components of the disclosure and cannot be understood as indicating or implying the relative importance of the components.

The above descriptions are only the illustrated embodiments of the disclosure, but the scope of the protection of the disclosure is not limited by the above embodiments. Any modification, equivalent replacement and improvement made by those skilled in the related field within the technical solutions of the disclosure and within the spirit and principles of the disclosure should fall within the scope of the protection of the disclosure.

What is claimed is:

1. An application method of a near-infrared fluorescent small-molecule probe, comprising:
using the near-infrared fluorescent small-molecule probe to prepare a medicine for targeting tumor tissues, in vivo near-infrared fluorescence imaging, and tumor photothermal therapy.

2. The application method according to claim 1, wherein the near-infrared fluorescent small-molecule probe is IR-780-F and a molecular formula of the IR-780-F is C₃₆H₃₈F₆ClN₂I with a structural formula expressed as follows:

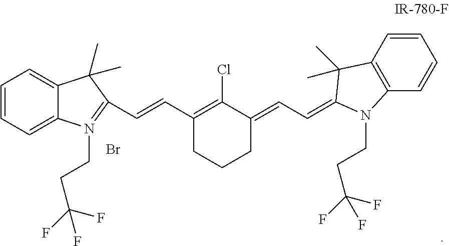

3. A testing kit for targeting tumor tissues, in vivo near-infrared fluorescence imaging, tumor photothermal and targeting breast carcinoma, comprising the near-infrared fluorescent small-molecule probe according to claim 2.

4. A synthesis method of a near-infrared fluorescent small-molecule probe, comprising:
step 1, adding carbon tetrabromide (CBr₄) and triphenylphosphine (PPh₃) into a 100 milliliters (mL) three-necked flask to obtain a solution, placing the 100 mL three-necked flask added with the solution in an ice-water mixture to make the solution cool down to 0 Celsius degree (° C.), and then slowly adding a 3,3,3-trifluoro-1-propanol (C₃H₅F₃O) reagent into the 100 mL three-necked flask added with the solution;
step 2, obtaining a first reaction solution until the solution in the step 1 to be a yellow viscous liquid after adding the 3,3,3-trifluoro-1-propanol reagent; heating and refluxing the 100 mL three-necked flask at 60° C. for 1 hour (h) after moving the 100 mL three-necked flask with the first reaction solution to a room temperature; cooling down the 100 mL three-necked flask and setting up a distillation apparatus, heating the distillation apparatus to vaporize a vaporized product, heating up to 100° C. to vaporize a liquid, and continuing to heat until no evaporated liquid to obtain a first colorless liquid compound;
step 3, dissolving the first colorless liquid compound, 2,3,3-trimethylindolenine (C₁₁H₁₃N) and potassium iodide (KI) into acetonitrile to heat to 150° C. in a closed container to obtain a first mixture, stirring the first mixture overnight for reaction to obtain a second reaction solution;
step 4, determining a reaction extent of the second reaction solution by using thin layer chromatography, wherein the thin layer chromatography displays less surplus of raw materials, the second reaction solution changes from light yellow to salmon pink; filtering the second reaction solution to obtain a first filtrate and a filter cake, washing the filter cake with the acetonitrile to obtain a second filtrate; concentrating the first filtrate and the second filtrate to obtain a first concentrated product, and performing silica gel column chromatography on the first concentrated product to obtain a second compound;
step 5, dissolving the second compound and 2-chloro-1-formyl-3-hydroxymethylenecyclohexene (C₈H₉O₂Cl) into a mixed solution of butyl alcohol and methylbenzene to obtain a second mixture; stirring the second mixture at 110° C. overnight for reaction, to obtain a third reaction solution; and
step 6, determining a reaction extent of the third reaction solution by using thin layer chromatography, wherein the thin layer chromatography displays no surplus of the raw materials, and a new sample spot is generated on a thin layer chromatography plate; and stopping reaction of the third reaction solution, concentrating and drying the third reaction solution to obtain a second concentrated product, and purifying the second concentrated product by the silica gel column chromatography to obtain a targeting product IR-780-F as the near-infrared fluorescent small-molecule probe.

5. The synthesis method according to claim 4, wherein in the step 1, an addition amount of the carbon tetrabromide is 13.95 grains (g) with a molar mass of 42.08 millimoles (mmol); an addition amount of the triphenylphosphine is 11.04 g with a molar mass of 42.08 mmol; and an addition amount of the 3,3,3-trifluoro-1-propanol is 4.0 g with a molar mass of 35.07 mmol.

6. The synthesis method according to claim 4, wherein in the step 2, a mass of the first colorless liquid compound is 0.75 g, taking a percentage of 12.1% in the first reaction solution.

7. The synthesis method according to claim 4, wherein in the step 3, an addition amount of the first colorless liquid compound is 0.65 g with a molar mass of 4.24 mmol; an addition amount of the 2,3,3-trimethylindolenine is 0.45 g with a molar mass of 2.83 mmol; an addition amount of the potassium iodide is 0.47 g with a molar mass of 2.83 mmol; a volume of the acetonitrile is 20 mL.

8. The synthesis method according to claim 4, wherein in the step 4, an developing reagent of the thin layer chromatography is a volume ratio of petroleum ether:ethyl acetate being 10:1; a condition for the silica gel column chromatography is a volume ratio of the petroleum ether: the ethyl acetate being a range from 100:1 to 5:1; and a mass of the second compound is 60 milligrams (mg), taking a percentage of 8.48% in the second reaction solution.

9. The synthesis method according to claim 4, wherein in the step 5, an addition amount of the second compound is 0.02 g with a molar ratio of 0.08 mmol; an addition amount of the 2-chloro-1-formyl-3-hydroxymethylenecyclohexene is 0.01 g with a molar ratio of 0.04 mmol; a ratio of the butyl alcohol: the methylbenzene in the mixed solution is 7:3, and a total volume of the mixed solution of the butyl alcohol and the methylbenzene is 2 mL.

10. The synthesis method according to claim 4, wherein in the step 6, the developing reagent of the thin layer chromatography is a volume ratio of the petroleum ether: the ethyl acetate being 10:1; and a condition for the purifying by the silica gel column chromatography is a volume ratio of the petroleum ether: the ethyl acetate being 1:1.
